(19) United States
(12) Patent Application Publication
(10) Pub. No.: US 2022/0223297 A1
(43) Pub. Date: Jul. 14, 2022

(54) DEVICE, METHOD AND PROGRAM PROVIDING REFERENCE INFORMATION FOR DISEASE DIAGNOSIS, BASED ON BODY TEMPERATURE

(71) Applicant: MOBILE DOCTOR CO., LTD., Seoul (KR)

(72) Inventors: Namsoo OH, Seongnam-si (KR); Myeongchan KIM, Anyang-si (KR); Sanghyun AHN, Cheonan-si (KR); Jae Won SHIN, Seoul (KR)

(73) Assignee: MOBILE DOCTOR CO., LTD., Seoul (KR)

(21) Appl. No.: 17/147,673

(22) Filed: Jan. 13, 2021

(30) Foreign Application Priority Data
Jan. 13, 2021 (KR) 10-2021-0004336

(51) Int. Cl. G16H 50/80; G16H 70/60; G16H 80/00; G16H 50/70

(52) U.S. Cl. CPC G16H 50/80; G16H 50/70; G16H 80/00; G16H 70/60

(57) ABSTRACT

The present disclosure relates to a device for providing reference information for disease diagnosis based on body temperature, and creates a group by searching for other subscribers related to context information and body temperature information of a specific subscriber, and calculates disease prevalence for the subscribers in the group, so that it is possible to calculate the prevalence of various diseases in the group related to a specific subscriber in numerical values.

### CROSS-REFERENCE TO RELATED APPLICATION

A claim for priority under 35 U.S.C. § 119 is made to Korean Patent Application No. 10-2021-0004336 filed on Jan. 13, 2021 in the Korean Intellectual Property Office, the entire contents of which are hereby incorporated by reference.

### BACKGROUND

### 1. Technical Field

The present disclosure provides reference information for disease diagnosis based on the body temperature information of a subscriber.

### 2. Description of Related Art

In general, when people develop symptoms such as a fever in their body, they visit a hospital to consult a doctor.

In addition, the doctor examines a patient, identifies symptoms, and makes a diagnosis of what disease the patient has.

At this time, since the doctor has to determine the patient's disease only by examining the patient, it is sometimes difficult to make an accurate diagnosis.

For example, if a pandemic disease is occurring in a specific area, a specific patient has a residence in the area and starts manifesting symptoms similar to the disease, it will be possible to make a more accurate and quick diagnosis for the patient.

However, a technology for generating and providing diagnosis assistance information for a patient in consideration of the patient's location information and the patient's situation as above is not currently disclosed to the public.

### RELATED ART DOCUMENT

### Patent Document

Korean Patent Application Publication No. 10-2020-0023029, Mar. 4, 2020

### SUMMARY OF THE INVENTION

Various aspects of the present disclosure are directed to creating a group by searching for other subscribers related to context information and body temperature information of a specific subscriber, and calculating disease prevalence for the subscribers in the group.

In addition, the present disclosure is directed to generating diagnosis assistance information for a specific subscriber based on the calculated disease prevalence and providing the medical staff of the subscriber therewith.

The aspects of the present disclosure are not limited to the above-mentioned aspects, and other aspects, which are not mentioned, will be clearly understood by those skilled in the art from the following description.

A device for providing reference information for disease diagnosis based on the body temperature according to an exemplary embodiment of the present disclosure includes a communication unit that communicates with two or more subscriber terminals, and a processor that searches for two or more subscribers related to context information and body temperature information of a specific subscriber among subscribers when the body temperature information of the specific subscriber is received from a terminal of the specific subscriber through the communication unit, creates a group including the searched subscribers, calculates a first disease prevalence for subscribers in the group based on a diagnosis history of the subscribers in the created group, and generates diagnosis assistance information for the specific subscriber based on the first disease prevalence.

In addition, the device may further include a database that stores diagnostic history information including context information and body temperature information for all subscribers.

In addition, the processor may recognize a code related to a disease from a diagnosis result image of a medical staff received from a subscriber terminal, and store a medical staff diagnosis result of the subscriber in the database based on the recognized result.

In addition, when a grouping condition is received from a medical staff terminal of the specific subscriber, the processor may search for two or more subscribers related to the grouping condition among the subscribers, and create a group including the searched subscribers.

In addition, when the predicted diagnosis name for the specific subscriber is input from a medical staff terminal of the specific subscriber, the processor calculates a first disease prevalence for the predicted diagnosis name for the subscribers in the group, and provides the calculated result to the medical staff terminal.

In addition, the processor may request the input of a prior questionnaire in a terminal of the specific subscriber before the specific subscriber visits a medical staff, analyze the prior questionnaire input and received from a terminal of the specific subscriber, generate a questionnaire analysis result, and generate diagnosis assistance information for the specific subscriber based on the calculated first disease prevalence and the questionnaire analysis result.

In addition, the processor may select a disease matching the questionnaire analysis result when there is a plurality of diseases exceeding a preset first prevalence value in the first prevalence calculation result, and generate diagnosis assistance information for the specific subscriber based on the first prevalence for the selected disease.

In addition, the processor may create a regional group by including subscribers having the same location information for each pre-divided area, calculate a second disease prevalence for the subscribers in each regional group at each preset period based on the body temperature information of the subscribers in the regional group and the medical staff diagnosis result, and provide a body temperature measurement request signal to a subscriber terminal having location information for the specific regional group when the second disease prevalence for a specific disease in a specific regional group exceeds a preset value.

In addition, the processor may determine that the subscriber has the location information for the specific regional group when at least one location information of residence location information of a subscriber and a subscriber family and preset main visiting area location information is related to the specific regional group.

In addition, the context information includes at least one condition among an age group condition, a gender condition, and a location information condition, and the processor may search for two or more subscribers related to body temperature information and at least one condition among the context information of the specific subscriber.

In addition, the processor may search for two or more subscribers with information related to the same age group and body temperature information as the specific subscriber when an age group condition is selected from the context information, search for two or more subscribers related to the same age group and body temperature information as the specific subscriber when a gender condition is selected from the context information, and search for two or more subscribers having the body temperature information and location information of the same area as the specific subscriber, or having location information within a certain distance from the location information of the specific subscriber when a location information condition is selected from the context information.

In addition, the body temperature information includes body temperature measurement location information, and the processor may determine location information of the specific subscriber in consideration of the time spent at the body temperature measurement location of the specific subscriber when the body temperature measurement location of the specific subscriber is different from the location information of the residence and main destination of the specific subscriber.

In addition, the communication unit may receive symptom information of the specific subscriber from a terminal of the specific subscriber, and the processor may select at least one condition of the context information based on the body temperature information and symptom information of the specific subscriber.

A method for providing reference information for disease diagnosis based on the body temperature according to an exemplary embodiment of the present disclosure includes searching for two or more subscribers related to context information and body temperature information of a specific subscriber among subscribers when the body temperature information of the specific subscriber is received from a terminal of the specific subscriber, creating a group including the searched subscribers, calculating a first disease prevalence for subscribers in the group based on a diagnosis history of subscribers in the created group, and generating diagnosis assistance information for the specific subscriber based on the first disease prevalence, wherein the computer communicates with two or more subscribers through a communication unit.

In addition to these embodiments, another method and system for implementing the present disclosure, and a computer-readable recording medium storing a computer program for executing the method may be further provided.

According to an exemplary embodiment of the present disclosure, a group is created by searching for other subscribers related to context information and body temperature information of a specific subscriber, and the disease prevalence for the subscribers in the group is calculated, so that it is possible to calculate the prevalence of various diseases in the group related to a specific subscriber in numerical values.

In addition, according to an exemplary embodiment of the present disclosure, diagnosis assistance information for a specific subscriber is generated based on the calculated disease prevalence and is provided to the medical staff of the subscriber, so that the medical staff can make a quick and accurate diagnosis for the subscriber.

The effects of the present disclosure are not limited to the above-mentioned effects, and other effects, which are not mentioned, will be clearly understood by those skilled in the art from the following description.

### BRIEF DESCRIPTION OF THE DRAWINGS

FIG. 1 is a block diagram of a system for providing reference information for disease diagnosis based on body temperature according to an embodiment of the present disclosure.

FIG. 2 is a flow diagram of a method for providing reference information for disease diagnosis based on body temperature according to an embodiment of the present disclosure.

FIG. 3 is a diagram illustrating the location of a residential area of subscriber #1 in area A divided into four areas.

FIG. 4 is a diagram illustrating a list of subscribers in area A3 corresponding to the residential area of subscriber #1.

FIG. 5 is a diagram illustrating an example of creating a group including subscribers selected in FIG. 4 and calculating prevalence using the same.

FIG. 6 is a diagram illustrating the prevalence value calculated using FIG. 5.

### DETAILED DESCRIPTION

Advantages and features of the present disclosure and methods for achieving them will be apparent from the embodiments described below in detail in conjunction with the accompanying drawings. However, the present disclosure is not limited to the embodiments disclosed below, but may be implemented in various different forms. The embodiments are provided so that this disclosure will be thorough and complete and will fully convey the scope of the present disclosure to those skilled in the technical field to which the present disclosure pertains. It is to be noted that the scope of the present disclosure is defined only by the claims.

The terminology used herein is for the purpose of describing embodiments and is not intended to limit the present disclosure. As used herein, the singular may be intended to include the plural forms as well, unless the context clearly indicates otherwise. The terms “comprises” and/or “comprising” used herein do not preclude the presence or addition of one or more other components, in addition to the mentioned components. Like reference numerals designate like components throughout the specification. As used herein, the term “and/or” includes each and all combinations of one or more of the mentioned components. It will be understood that, although the terms “first”, “second”, etc., may be used herein to describe various components, these components should not be limited by these terms. These terms are only used to distinguish one component from another component. Accordingly, a first component mentioned below could be termed a second component without departing from the technical ideas of the present disclosure.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

Hereinafter, exemplary embodiments of the present disclosure will be described in detail with reference to the accompanying drawings.

Prior to the description, the meanings of the terms used in the present specification will be described briefly. However, it should be noted that the description of terms is used to help the understanding of the present specification, but is not to be used to limit the technical spirit of the present disclosure in the case where the limitative details of the present disclosure are not explicitly described.

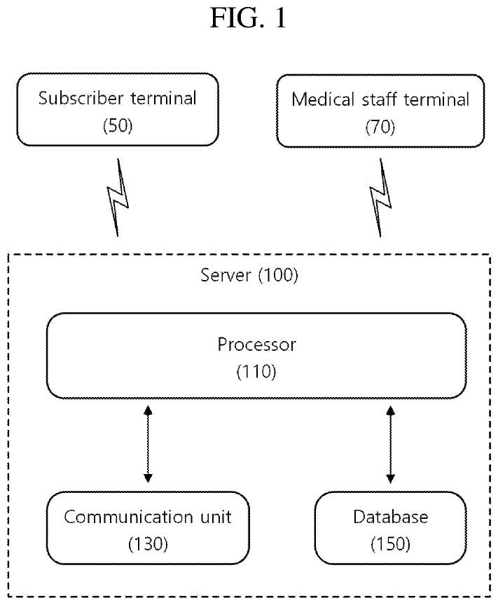

FIG. 1 is a block diagram of a system 10 for providing reference information for disease diagnosis based on body temperature according to an embodiment of the present disclosure.

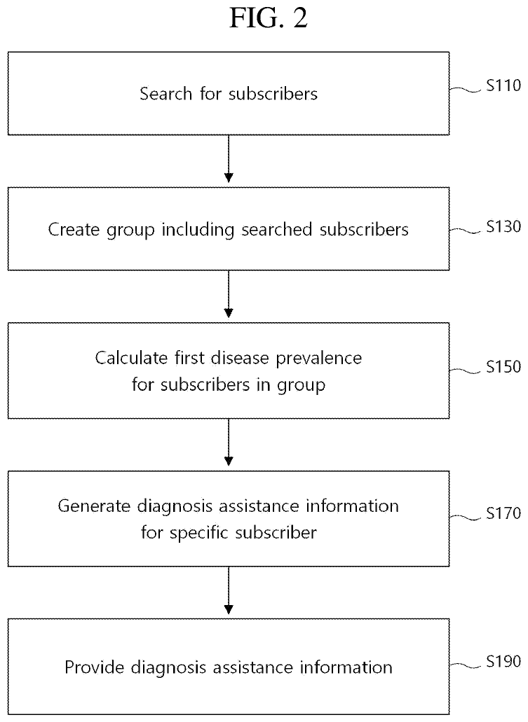

FIG. 2 is a flow diagram of a method for providing reference information for disease diagnosis based on body temperature according to an embodiment of the present disclosure.

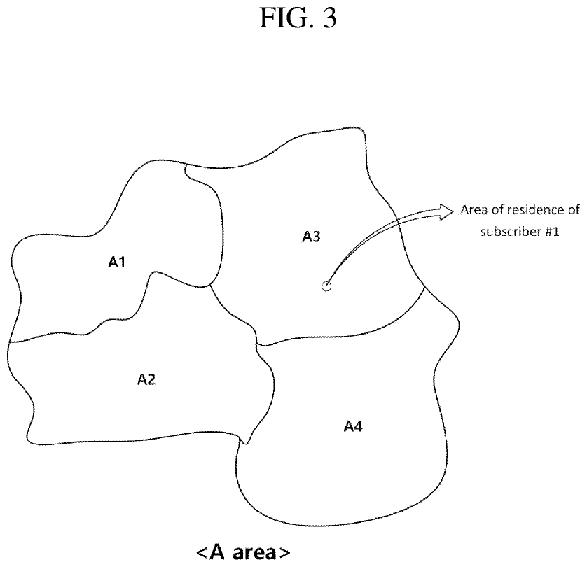

FIGS. 3 to 6 are various exemplary diagrams to help explain the system 10 for providing reference information for disease diagnosis based on body temperature according to an embodiment of the present disclosure.

Referring to FIG. 1, the system 10 for providing reference information for disease diagnosis based on body temperature according to an embodiment of the present disclosure includes a server 100, a subscriber terminal 50, and a medical staff terminal 70, and the server 100 includes a processor 110, a communication unit 130, and a database 150.

However, in some embodiments, the server 100 may include a smaller number of components or more components than the components illustrated in FIG. 1.

The system 10 for providing reference information for disease diagnosis based on body temperature according to an embodiment of the present disclosure is performed by a device, and in FIGS. 1 and 2 and the embodiments to be described below, the device is described as being implemented as the server 100.

In addition, a device for providing reference information for disease diagnosis based on body temperature may be implemented as a computer or information processing means.

The server 100 may provide a diagnosis assistance information generation service, and may provide a service through a service application.

Accordingly, a general subscriber who subscribes to the service installs a service application on a terminal and logs in as a general subscriber to use the service.

In addition, a medical staff subscriber who subscribes to the service installs a service application on a terminal and logs in as a medical staff subscriber to use the service.

The difference between a general subscriber and a medical staff subscriber is that the type of service applications is different from each other or login information may be set differently.

In an embodiment of the present disclosure, devices such as a smart phone, a tablet PC, a notebook, and a computer may be applied to a terminal.

The communication unit 130 communicates with two or more subscriber terminals 50 and medical staff terminals 70.

In detail, the communication unit 130 may communicate with the terminals of all subscribers who have subscribed to the service, and may also communicate with the medical staff terminal 70 to provide the finally generated diagnosis assistance information to a medical staff.

When a subscriber subscribes to a service through the communication unit 130, the server 100 may receive personal information such as age and gender through the terminal 50, and may receive location information.

In detail, the location information includes location information of a subscriber's residence area and location information of subscriber's main visiting area(s), and the main visiting areas may correspond to mainly visited places other than a subscriber's residence, such as a subscriber's school, institute, and workplace.

In addition, the server 100 may receive the information of subscriber's family from a subscriber and store it in the database 150.

The processor 110 is responsible for controlling all configurations in the server 100 and may provide a service by executing an algorithm stored in the database 150.

In addition, the processor 110 stores data such as personal information and body temperature information input and received from the subscriber terminal 50 in the database 150.

In addition, the processor 110 stores diagnostic history information including context information and body temperature information for all subscribers in the database 150.

In one embodiment, the processor 110 may receive body temperature information and medical staff diagnosis results of subscribers who visit a hospital after fever and consult a doctor, and may store them in the database 150.

The server 100 may directly receive a medical staff diagnosis result for a subscriber from the medical staff terminal 70, and may receive the result from the subscriber terminal 50.

In addition, the processor 110 may use image recognition technology when a diagnosis result is received from the subscriber terminal 50.

Specifically, the processor 110 recognizes a code related to a disease from the diagnosis result image of the medical staff received from the subscriber terminal 50, determines the medical staff diagnosis result of the subscriber based on the recognized result, and stores the result in the database 150.

For example, when a medical staff document on which a disease code, such as a medical certificate, a medical treatment confirmation, and an outpatient confirmation, is described, is filmed and received from the subscriber terminal 50, the processor 110 may check the disease code from the image and determine the subscriber's disease.

However, it does not mean that all subscribers have been diagnosed with a disease, but they may receive a result diagnosed as not having a disease after their visit to a hospital for symptoms.

As described above, each configuration of the system 10 for providing reference information for disease diagnosis based on body temperature according to an embodiment of the present disclosure has been schematically described.

Hereinafter, the system 10 for providing reference information for disease diagnosis based on body temperature according to an embodiment of the present disclosure will be described in more detail with reference to FIG. 2.

In addition, when describing each embodiment, it will be described with reference to FIGS. 3 to 6

First, when the server 100 receives body temperature information of a specific subscriber from the terminal 50 of a specific subscriber through the communication unit 130, the server 100 searches for two or more subscribers related to the context information and body temperature information of a specific subscriber among subscribers. (S110)

After S110, the processor 110 creates a group including the subscribers searched in S110. (S130)

The server 100 may perform S110 when it is determined that the body temperature value is not the normal body temperature from the body temperature information received from the terminal 50 of a specific subscriber.

In addition, the server 100 may perform S110 when an assistance diagnostic request signal is received from the terminal 50 of a specific subscriber and a body temperature value is received.

For example, if a specific subscriber feels feverish in his or her body, it is possible to determine that he or she may have a disease, input a body temperature value through a terminal, and request an assistance diagnosis.

In an embodiment of the present disclosure, the server 100 may receive body temperature information including a body temperature value from the subscriber terminal 50, and the subscriber may directly input the body temperature value in the terminal.

However, it is not limited thereto, and a terminal may register body temperature information by automatically receiving the measured body temperature value when body temperature measurement is detected from an interlocked thermometer.

For example, a subscriber may interlink his or her own temperature measuring device with a terminal.

In addition, the server 100 may provide a body temperature measurement request signal to a subscriber terminals according to a preset algorithm, and a detailed description thereof will be described later.

The context information may include various conditions, and includes an age group condition, a gender condition, and a location information condition.

When an age group condition is selected from the context information, the processor 110 searches for two or more subscribers related to the same age group and body temperature information as the specific subscriber.

When a gender condition is selected from the context information, the processor 110 searches for two or more subscribers related to the same gender and body temperature information as the specific subscriber.

When a location information condition is selected from the context information, the processor 110 searches for two or more subscribers having the body temperature information and location information of the same area as the specific subscriber, or having location information within a certain distance from the location information of the specific subscriber.

The processor 110 may select at least one condition from the context information of a specific subscriber and search for two or more subscribers related to the selected condition and body temperature information.

In this way, the server 100 searches for subscribers in consideration of not only body temperature information but also the situation of a specific subscriber, so that it is possible to search for subscribers in a situation similar to that of a specific subscriber among other subscribers who are running a fever or have a history of occurrence.

In some embodiments, the context information may include at least one of an oxygen saturation level and a blood pressure level.

In addition, when selecting at least one condition from the context information of a specific subscriber, the processor 110 may further include at least one of an oxygen saturation level or a blood pressure level as a condition, and search for two or more subscribers related to the selected condition and body temperature information.

For example, as a result of analyzing symptoms of a specific user, when a condition accompanying a suspected disease includes a specific oxygen saturation level or a specific blood pressure level, the processor 110 further includes at least one condition of an oxygen saturation level or a blood pressure level so as to search for subscribers.

In detail, in the above case, the processor 110 may search for subscribers who have numerical value records within a certain range from the oxygen saturation level of a specific subscriber or within a certain range from the blood pressure level of a specific subscriber.

In an embodiment, the body temperature information includes body temperature measurement location information.

In addition, when the body temperature measurement location of a specific subscriber is different from the preset residence and main destination of the specific subscriber, the processor 110 may determine the location information of the specific subscriber in consideration of the time spent at the body temperature measurement location of the specific subscriber.

In an embodiment of the present disclosure, the server 100 may receive symptom information of a specific subscriber from the terminal 50 of a specific subscriber through the communication unit 130.

In addition, the processor 110 selects at least one condition from the context information based on the body temperature information and symptom information of a specific subscriber.

After the processor 110 analyzes the body temperature information and symptom information of a specific subscriber, if it is determined that diseases A and B are suspected, in order to calculate the prevalence with diseases A and B as the main analysis targets, it means selecting conditions that can easily search for other subscribers corresponding to diseases A and B.

For example, in order to easily search for other subscribers corresponding to diseases A and B, if the processor 110 determines that it is the most probable to search for a child aged 5 to 10 in the same area as a specific subscriber, the location information condition of the same area and the age group condition of 5-10 years old are selected.

However, the processor 110 is not limited as described above to select a condition, and the condition may be directly input from a medical staff.

In detail, when a grouping condition is received from the medical staff terminal 70 of a specific subscriber, the processor 110 may search for two or more subscribers related to the grouping condition among subscribers, and create a group including the searched subscribers.

After S130, the processor 110 calculates a first disease prevalence for the subscribers in the group generated in S130. (S150)

After S150, diagnosis assistance information for a specific subscriber is generated based on the first disease prevalence. (S170)

After S170, the processor 110 provides the diagnosis assistance information generated in S170 through the communication unit 130 to the medical staff terminal 70 of a specific subscriber. (S190)

In detail, the processor 110 inquire into the diagnosis history of the subscribers in the created group, and calculates a first disease prevalence for the subscribers in the group based thereon.

The processor 110 calculates a first disease prevalence for each of at least one disease for the subscribers in the group.

The processor 110 may calculate the first disease prevalence in numerical values.

When the diagnosis history of the subscribers in the group is inquired, it is possible to check the diagnosis result (positive or negative) for a specific disease of each subscriber, and to calculate the first disease prevalence value by using statistical information about this.

For example, if there are 10,000 subscribers in the group, and if it is inquired that among them, 7,000 people have had or have disease A, and 500 people have had or have disease B, the processor 110 may calculate that the first disease prevalence value for disease A is much higher than the first disease prevalence value for disease B.

As described above, the processor 110 may inquire the disease diagnosis history of subscribers in the group, and calculate the first disease prevalence based on the number of positive and negative diagnoses.

Hereinafter, an example will be given with reference to FIGS. 3 to 6.

FIG. 3 is a diagram illustrating the location of a residential area of subscriber #1 in area A divided into four areas.

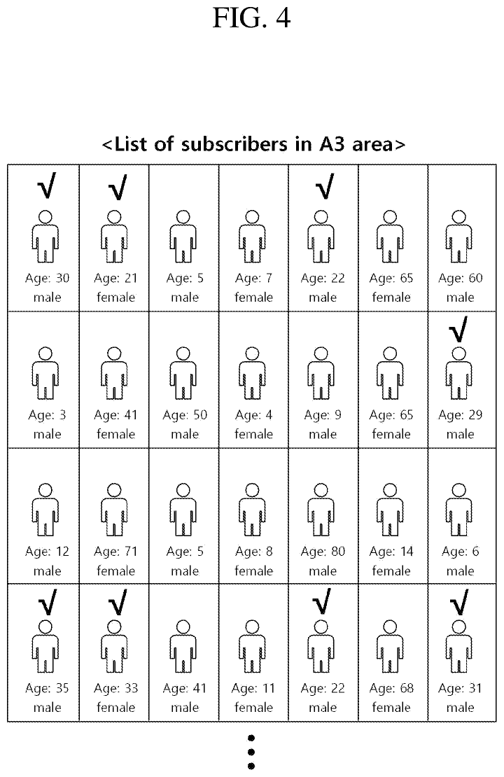

FIG. 4 is a diagram illustrating a list of subscribers in area A3 corresponding to the residential area of subscriber #1.

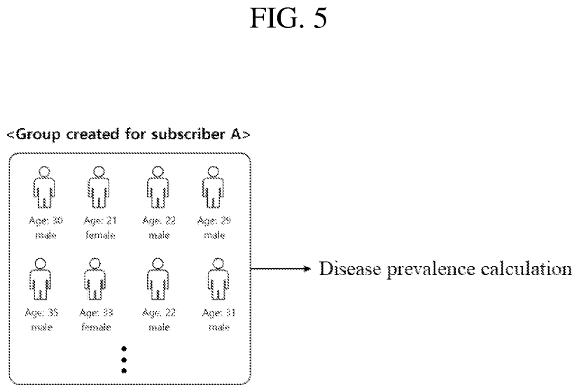

FIG. 5 is a diagram illustrating an example of creating a group including subscribers selected in FIG. 4 and calculating prevalence using the same.

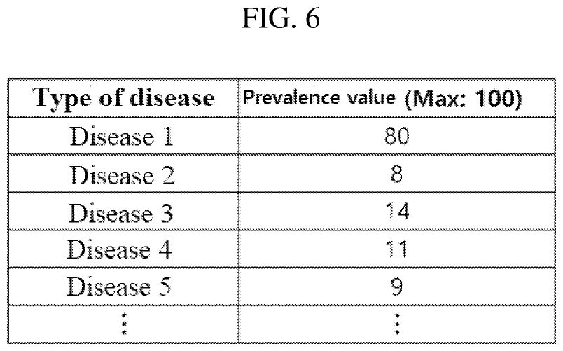

FIG. 6 is a diagram illustrating the prevalence value calculated using FIG. 5.

The processor 110 checks that the residential area of subscriber #1 is located within A3, searches for subscribers having location information in A3 area, and searches and lists the subscribers as illustrated in FIG. 4.

Here, when the processor 110 applies the age group condition of 20-30 years old in the context information of subscriber #1, subscribers in their 20 s to 30 s in A3 area are searched as illustrated in FIG. 4, and a group including the searched subscribers is created as illustrated in FIG. 5.

Next, the processor 110 calculates the first disease prevalence of each of at least one disease for the subscribers in the group based on the diagnosis history of the subscribers in the created group. This result is illustrated in FIG. 6.

Referring to FIG. 6, it is assumed that there are a total of 1,000 people in the number of subscribers in the created group, there are 800 subscribers diagnosed with disease 1, there are 80 subscribers diagnosed with disease 2, there are 140 subscribers diagnosed with disease 3, there are 110 subscribers diagnosed with disease 4, and there are 90 subscribers diagnosed with disease 5, and it is assumed that the first disease prevalence value ranges from 0 to 100.

Assuming as above, the processor 110 may calculate the first disease prevalence as disease 1 as 80, disease 2 as 8, disease 3 as 14, disease 4 as 11, and disease 5 as 9.

In addition, the processor 110 generates diagnosis assistance information for a specific subscriber based on the generated first disease prevalence value, and provides it to the medical staff terminal 70 set as a medical staff of a specific subscriber.

The processor 110 providing diagnosis assistance information to the medical staff terminal 70 is only an example, and any person who can check the status of a specific subscriber other than a medical staff can apply anything.

A medical staff receives and checks the diagnosis assistance information from the server 100, and may obtain help in diagnosing a specific subscriber.

In the embodiments of the present disclosure, the type of disease is not limited, but any infectious or epidemic disease may be applied.

In detail, diseases that may be applied in the embodiments of the present disclosure typically include flu and febrile diseases, and in addition, diseases related to fever and infectious and epidemic diseases may be included.

In addition, in order to determine the condition according to the context information in S110, the database 150 of the server 100 may store information on a prevalence, an age group who is infected, a gender, and a region for each disease.

In one embodiment, when the predicted diagnosis name for a specific subscriber is received from the medical staff terminal 70, the processor 110 calculates a first disease prevalence for the predicted diagnosis name for the subscribers in the group, and provides the calculated result to the medical staff terminal (70).

The processor 110 may generate diagnosis assistance information by calculating the first disease prevalence values for all diseases, but as described above, the processor 110 may receive the predicted diagnosis name from a medical staff and calculate the first disease prevalence to provide diagnosis assistance information.

This may be used in situations in which a medical staff checks symptom information and information on a medical examination by interview before diagnosing a specific member and a specific disease is clearly suspected, and this may also be used when a medical staff wishes to calculate in detail a first disease prevalence for a specific disease suspected in the process of diagnosing a specific subscriber.

In one embodiment, the processor 110 may request the input of a prior questionnaire in the terminal 50 of a specific subscriber before the specific subscriber visits a medical staff.

The prior questionnaire is already set and stored in the database 150, and the processor 110 provides it to a specific subscriber terminal 50 to request input through a service application, so that a specific subscriber can input the prior questionnaire.

The processor 110 generates a result of analyzing the questionnaire by analyzing the prior questionnaire received and input from the terminal 50 of a specific subscriber.

In addition, the processor 110 may generate diagnosis assistance information for a specific subscriber based on the calculated first disease prevalence and questionnaire analysis result and provide it to the medical staff terminal 70.

For example, when the first prevalence values for two or more diseases are similarly calculated, the processor 110 may generate diagnosis assistance information by evaluating the priority among two or more diseases using the questionnaire analysis result.

Through this configuration, when similar first prevalence values are calculated for multiple diseases, a medical staff can more quickly and accurately diagnose a specific subscribers by setting priorities for multiple diseases more objectively using the results of a prior medical examination by interview.

In one embodiment, the processor 110 may select a disease matching the result of analyzing the questionnaire when there are a plurality of diseases exceeding the preset first prevalence value in the first prevalence calculation result, and generate diagnosis assistance information for a specific subscriber based on a first prevalence for the selected disease.

In one embodiment, the server 100 may request the subscribers who have subscribed to the service to input body temperature information by measuring their body temperature every preset period.

In addition, the processor 110 may analyze the situation and body temperature information of subscribers in real time or periodically, and request the subscribers to measure the body temperature when a specific condition is satisfied.

In detail, the processor 110 creates a regional group by including subscribers having the same location information for each pre-divided area.

The processor 110 calculates a second disease prevalence for the subscribers in each regional group at each preset period based on the body temperature information of the subscribers in the regional group and the medical staff diagnosis result.

In addition, when the processor 110 detects that the second disease prevalence for a specific disease in a specific regional group exceeds a preset value, the processor 110 may provide a body temperature measurement request signal to the subscriber terminal (50) having location information for the regional group.

The processor 110 may determine that the subscriber has the location information for the specific regional group when at least one of the location information of a subscriber and a subscriber's family residence and the preset main visiting area location information is related to a specific regional group.

In one embodiment, the processor 110 may inquire the personal information of a specific subscriber and, when there is a family member, may inquire family information including an age and gender of each family member.

In addition, the processor 110 may calculate at least one of the possibility of infection of a family member's disease and the possibility of infection from a family member and include the same in the diagnosis assistance information.

In detail, when the first disease prevalence of each disease is calculated, the processor 110 searches for information on infection characteristics of each disease to understand an age group and gender in which each disease is prevalent.

In addition, based thereon, when a specific subscriber is diagnosed as being infected with a specific disease, the processor 110 may calculate a probability that a family member has been infected with the disease by a specific subscriber.

In addition, based thereon, when a specific subscriber is diagnosed as being infected with a specific disease, the processor 110 may calculate a probability that a specific subscriber has been infected with the disease by a family member.

The method according to an embodiment of the present disclosure described above may be implemented as a program (or an application) to be executed in combination with a server, which is hardware, and stored in a medium.

The above-described program may include a code encoded by a computer language such as C, C++, JAVA, or a machine language, which a processor (CPU) of the computer can read through a device interface of the computer, such that the computer reads the program and performs the methods implemented with the program. The code may include functional codes associated with the function that defines functions necessary to perform the methods, and may include a control code associated with an execution procedure necessary for the processor of the computer to perform the functions in a predetermined procedure. Furthermore, the code may further include additional information necessary for the processor of the computer to perform the functions or a memory reference-related code associated with the location (address) of the internal or external memory of the computer, at which the media needs to be referred. In addition, when the processor of the computer needs to communicate with any other remote computer or any other remote server to perform the functions, the code may further include a communication-related code associated with how to communicate with any other remote computer or server using the communication module of the computer, and what information or media should be transmitted or received during communication.

The storing media may mean the media that does not store data for a short period of time such as a register, a cache, a memory, or the like but semi-permanently stores to be read by the device. Specifically, for example, the storing media include, but are not limited to, ROM, RAM, CD-ROM, magnetic tape, floppy disk, optical data storage device, and the like. That is, the program may be stored in various recording media on various servers that the computer can access, or various recording media on the computer of the user. In addition, the media may be distributed to a computer system connected to a network, and a computer-readable code may be stored in a distribution manner.

The steps of a method or algorithm described in connection with the embodiments of the present disclosure may be embodied directly in hardware, in a software module executed by hardware, or in a combination thereof. The software module may reside on a Random Access Memory (RAM), a Read Only Memory (ROM), an Erasable Programmable ROM (EPROM), an Electrically Erasable Programmable ROM (EEPROM), a Flash memory, a hard disk, a removable disk, a CD-ROM, or a computer readable recording medium in any form well known in the technical field to which the present disclosure pertains.

Although the embodiments of the present disclosure have been described with reference to the attached drawings, those skilled in the technical field to which the present disclosure pertains will understand that the present disclosure may be practiced in other detailed forms without departing from the technical spirit or essential features of the present disclosure. Therefore, it should be understood that the above-described embodiments are exemplary in all aspects rather than being restrictive.

### DESCRIPTION OF SYMBOLS

10: diagnosis assistance information generation system
50: subscriber terminal
70: medical staff terminal
100: server
110: processor
130: communication unit
150: database

What is claimed is:

1. A device for providing reference information for disease diagnosis based on body temperature, the device comprising:
a communication unit that communicates with two or more subscriber terminals; and
a processor that searches for two or more subscribers related to context information and body temperature information of a specific subscriber among subscribers when the body temperature information of the specific subscriber is received from a terminal of the specific subscriber through the communication unit, creates a group including the searched subscribers, calculates a first disease prevalence for subscribers in the group based on a diagnosis history of the subscribers in the created group, and generates diagnosis assistance information for the specific subscriber based on the first disease prevalence.

2. The device of claim 1, further comprising a database that stores diagnostic history information including context information and body temperature information for all subscribers.

3. The device of claim 2, wherein the processor recognizes a code related to a disease from a diagnosis result image of a medical staff received from a subscriber terminal, and stores a medical staff diagnosis result of the subscriber in the database based on the recognized result.

4. The device of claim 1, wherein, when a grouping condition is received from a medical staff terminal of the specific subscriber, the processor searches for two or more subscribers related to the grouping condition among the subscribers, and creates a group including the searched subscribers.

5. The device of claim 1, wherein, when the predicted diagnosis name for the specific subscriber is received from a medical staff terminal, the processor calculates a first disease prevalence for the predicted diagnosis name for the subscribers in the group, and provides the calculated result to the medical staff terminal.

6. The device of claim 1, wherein the processor requests input of a prior questionnaire in a terminal of the specific subscriber before the specific subscriber visits a medical staff, analyzes the prior questionnaire input and received from a terminal of the specific subscriber and generates a questionnaire analysis result, and generates diagnosis assistance information for the specific subscriber based on the calculated first disease prevalence and the questionnaire analysis result.

7. The device of claim 6, wherein, when there is a plurality of diseases exceeding a preset first prevalence value in the first prevalence calculation result, the processor selects a disease matching the questionnaire analysis result, and generates diagnosis assistance information for the specific subscriber based on a first prevalence for the selected disease.

8. The device of claim 1, wherein the processor creates a regional group by including subscribers having the same location information for each pre-divided area, calculates a second disease prevalence for the subscribers in each regional group at each preset period based on the body temperature information of the subscribers in the regional group and the medical staff diagnosis result, and provides a body temperature measurement request signal to a subscriber terminal having location information for the specific regional group when the second disease prevalence for a specific disease in a specific regional group exceeds a preset value.

9. The device of claim 8, wherein, when at least one location information of residence location information of a subscriber and a subscriber family and preset main visiting area location information is related to the specific regional group, the processor determines that the subscriber has the location information for the specific regional group.

10. The device of claim 1, wherein the context information includes at least one condition among an age group condition, a gender condition, and a location information condition, and wherein the processor searches for two or more subscribers related to body temperature information and at least one condition among the context information of the specific subscriber.

11. The device of claim 10, wherein the processor searches for two or more subscribers with information related to the same age group and body temperature information as the specific subscriber when an age group condition is selected from the context information, searches for two or more subscribers related to the same age group and body temperature information as the specific subscriber when a gender condition is selected from the context information, and searches for two or more subscribers having the body temperature information and location information of the same area as the specific subscriber, or having location information within a certain distance from the location information of the specific subscriber when a location information condition is selected from the context information.

12. The device of claim 11, wherein the body temperature information includes body temperature measurement location information, and wherein, when a body temperature measurement location of the specific subscriber is different from location information of a residence and main destination of the specific subscriber, the processor determines location information of the specific subscriber in consideration of the time spent at the body temperature measurement location of the specific subscriber.

13. The device of claim 10, wherein the communication unit receives symptom information of the specific subscriber from a terminal of the specific subscriber, and wherein the processor selects at least one condition of the context information based on body temperature information and symptom information of the specific subscriber.

14. The device of claim 1, wherein the context information further comprises biometric information including at least one of oxygen saturation and blood pressure.

15. The device of claim 1, wherein, when the specific subscriber has a family member, the processor inquires family information including an age and gender of each family member, and calculates at least one of the possibility of infection of a family member's disease and the possibility of infection from a family member and includes the same in the diagnosis assistance information.

16. A method for providing reference information for disease diagnosis based on body temperature, performed by a computer, the method comprising:
searching for two or more subscribers related to context information and body temperature information of a specific subscriber among subscribers when the body temperature information of the specific subscriber is received from a terminal of the specific subscriber;
creating a group including the searched subscribers;
calculating a first disease prevalence for subscribers in the group based on a diagnosis history of subscribers in the created group; and
generating diagnosis assistance information for the specific subscriber based on the first disease prevalence,
wherein the computer communicates with two or more subscribers through a communication unit.

17. A recording medium in which a program for executing the method of claim 16 is stored in combination with a computer as hardware.
